(19) United States
(12) Patent Application Publication
(10) Pub. No.: US 2013/0116654 A1
(43) Pub. Date: May 9, 2013

(54) Aortic Occlusion Catheter

(71) Applicants: Mark Dehdashtian, Irvine, CA (US); Teodoro S. Jimenez, JR., Irvine, CA (US); RB Hayes, Woods Cross, UT (US); Amanda Larsen, Sandy, UT (US); Edwin Schulting, Roden (NL); Lex Boudewijn, Roden (NZ)

(72) Inventors: Mark Dehdashtian, Irvine, CA (US); Teodoro S. Jimenez, JR., Irvine, CA (US); RB Hayes, Woods Cross, UT (US); Amanda Larsen, Sandy, UT (US); Edwin Schulting, Roden (NL); Lex Boudewijn, Roden (NZ)

(73) Assignee: Edwards Lifesciences LLC, Irvine, CA (US)

(21) Appl. No.: 13/291,920

(22) Filed: Nov. 8, 2011

(51) Int. Cl. A61M 25/10

(52) U.S. Cl. 604/509; 604/96.01

(57) ABSTRACT

An cardioplegic fluid delivery catheter includes an expandable member for occluding the ascending aorta of a patient. A length of the catheter allows the distal end to be within the ascending aorta while the proximal end extends from a peripheral artery. The delivery catheter has a multi-lumen construction with a primary lumen extending configured to allow a cardioplegic fluid to be delivered to the aorta. Secondary lumens provide for balloon inflation and aortic root pressure monitoring. The delivery catheter includes a shaft having a pre-determined curve profile at a distal end of the delivery catheter. The pre-determined curve profile generally corresponds to the curve of the bottom surface of the aortic arch. The shaft may be eccentric to the expandable member such that retraction of the shaft causes a distal tip to be parallel within the ascending aorta.

### BACKGROUND

1. Field of the Invention

This invention relates generally to devices and techniques for performing cardiac procedures and particularly to catheter systems and methods for inducing cardioplegic arrest to facilitate the performance of cardiac procedures.

2. The Related Technology

Known techniques for performing major surgeries such as coronary artery bypass grafting and heart valve repair and replacement have generally required open access to the thoracic cavity through a large open wound, known as a thoracotomy. Typically, the sternum is cut longitudinally (i.e., a median sternotomy), providing access between opposing halves of the anterior portion of the rib cage to the heart and other thoracic vessels and organs. An alternate method of entering the chest is via a lateral thoracotomy, in which an incision, typically 10 cm to 20 cm in length, is made between two ribs. A portion of one or more ribs may be permanently removed to optimize access.

In procedures requiring a median sternotomy or other type of thoracotomy, the ascending aorta is readily accessible for placement of an external cross-clamp, and for introduction of a cardioplegic fluid delivery cannula and venting cannula through the aortic wall. However, such surgery often entails weeks of hospitalization and months of recuperation time, in addition to the pain and trauma suffered by the patient. Moreover, while the average mortality rate associated with this type of procedure is about two to fifteen percent for first-time surgery, mortality and morbidity are significantly increased for reoperation. Further, significant complications may result from such procedures. For example, application of an external cross-clamp to a calcified or atheromatous aorta may cause the release of emboli into the brachiocephalic, carotid or subclavian arteries with serious consequences such as strokes.

Methods and devices are therefore needed for isolating the heart and coronary arteries from the remainder of the arterial system, arresting cardiac function, and establishing cardiopulmonary bypass without the open-chest access provided by a median sternotomy or other type of thoracotomy. In particular, methods and devices are needed which facilitate the delivery of cardioplegia sufficiently to allow the heart to be placed under cardioplegic arrest with full cardiopulmonary bypass, without requiring open-chest access to the heart and without requiring an incision or puncture in the aorta, in the pulmonary artery, or in the heart wall. Embodiments of the present disclosure satisfy these and other needs.

### SUMMARY OF THE INVENTION

The present disclosure is directed to methods, systems, assemblies, and apparatus relating to an antegrade cardioplegia delivery catheter. More particularly, embodiments herein relate to a catheter, and methods and systems in which it is used, particularly related to performing cardiac procedures in which the catheter can be used to occlude all or a portion of the aorta.

In at least some embodiments, an antegrade cardioplegia delivery catheter is described. An example antegrade cardioplegia delivery catheter may be advanced into an aorta of a patient's heart for antegrade delivery of a fluid. Exemplary delivery catheters may include an elongated shaft having a proximal end and a distal end, with the elongated shaft having sufficient length and flexibility so that the proximal end may extend intraluminally through a patient's femoral or other artery when the distal end is positioned in the aorta of a patient. The elongated shaft can include multiple lumens, including at least a primary lumen configured to receive antegrade cardioplegia. One or more secondary lumens may also be included and can be provided for expanding an expandable member, measuring a pressure (e.g., aortic root pressure) in the heart of the patient, or passing a core element along all or a portion of the length of the elongated shaft. The core element may be configured to define a predetermined shape that causes the elongated shaft to flex to a conforming shape that approximates a curvature of a lower or bottom surface of the patient's aortic arch.

In some embodiments, a device for occluding a patient's ascending aorta includes a hub having one or more ports. An elongated body having opposing proximal and distal ends may be connected to the hub. The elongated body can define one or more lumens extending at least partially between the proximal and distal ends of the elongated body. Each of the one or more lumens may be in fluid communication with at least one of the one or more ports of the hub. The elongated body may have a pre-configured, selectively actuated curve profile at least proximate the distal end of the elongated body. An expandable member may be at the distal end of the elongated body and selectively changeable between expanded and contracted states.

In some embodiments, the elongated body is a multi-lumen extrusion, and can be coil-less and/or lack wire reinforcement. A core may extend through all or a portion of the elongated body, and can define the pre-configured curve profile. The core may be a memory material selectively actuated by, for instance, a temperature such as body temperature. In some embodiments, the pre-configured curve profile defines a radius of curvature of about eighteen millimeters plus-or-minus five percent.

In another embodiment, a device for occluding a patient's ascending aorta includes a hub and an elongated shaft connected to the hub. The elongated shaft has proximal and distal ends, and defines multiple lumens therebetween. An expandable member may be at least proximate the distal end of the elongated shaft, and selectively changeable between expanded and contracted states. In the expanded state, the expandable member may define an eccentric lumen with upper and lower surfaces of differing lengths.

In some embodiments, the offset shaft defines an eccentricity between about thirty and about seventy percent relative to the central axis. The expandable member may further be substantially symmetrical about a first central axis that separates distal and proximal portions of the expandable member, and substantially asymmetrical about a second central axis extending between the distal and proximal portions of the expandable member, and which is about perpendicular to the first central axis. Edges of the expandable member may be inclined at an angle between about thirteen degrees and about twenty degrees. In still other embodiments, a distal tip of the shaft is substantially enclosed within the expandable member.

In another embodiment, a method of delivering antegrade cardioplegic fluid to a heart of a patient includes introducing at least a distal end of an antegrade cardioplegic delivery catheter into a peripheral artery of the patient. The distal end of the antegrade cardioplegic delivery catheter may be advanced from the peripheral artery into an ascending aorta of the heart of the patient. Such advancement may include changing a curve profile of the delivery catheter to a predetermined curve profile configured to generally correspond to a bottom surface of an aortic arch of the patient. The ascending aorta may be occluded using an occlusion device, and a distal tip of the antegrade cardioplegic delivery catheter can be positioned and/or maintained in a generally parallel alignment within the ascending aorta. Fluid can be delivered to the heart through a lumen of the delivery catheter.

In accordance with one embodiment, the fluid is delivered through an opening in the distal tip of the catheter. Occlusion of the ascending aorta may also include removing slack within the antegrade cardioplegic delivery catheter. Such slack may be less than about three centimeters. Changing the curve profile may include a memory material automatically responding to a body temperature of the patient to return to a remembered curvature.

These and other advantages of the present disclosure will become more apparent from the following detailed description when taken in conjunction with the accompanying exemplary drawings. These and other aspects and features of the present disclosure will become more fully apparent from the following description and appended claims.

### BRIEF DESCRIPTION OF THE FIGURES

To further clarify various aspects of embodiments of the present disclosure, a more particular description of the certain embodiments will be made by reference to various aspects of the appended drawings. It is appreciated that these drawings depict only typical embodiments of the present disclosure and are therefore not to be considered limiting of the scope of the disclosure. Moreover, while the figures may be drawn to scale for some embodiments, the figures are not necessarily drawn to scale for all embodiments. Embodiments of the present disclosure will be described and explained with additional specificity and detail through the use of the accompanying drawings in which:

FIG. 1 schematically illustrates a cardiac access system according to one example embodiment of the present disclosure;

FIG. 2 is an enlarged partial section view of the cardiac access system of FIG. 1, particularly illustrating an occluding catheter disposed within the ascending aorta;

FIG. 3 is a transverse cross-sectional view of the exemplary occluding catheter of FIG. 2;

FIG. 4A is a side perspective view of an exemplary antegrade cardioplegia delivery catheter according to one aspect of the present disclosure;

FIG. 4B is side view of the distal end of the antegrade cardioplegia delivery catheter of FIG. 5A, the distal end including an expandable member and an atraumatic tip;

FIG. 4C is a cross-sectional view of a catheter shaft of the antegrade cardioplegia delivery catheter of FIG. 4A;

FIG. 4D is an isometric cross-sectional view of the atraumatic tip of the antegrade cardioplegia delivery catheter of FIG. 4A;

FIGS. 5A-5C illustrate partial cross-sectional views of the distal end of an antegrade cardioplegia delivery catheter used to occlude a portion of the ascending aorta, the delivery catheter having a concentric balloon and a curved shaft facilitating occlusion of the ascending aorta; and

FIGS. 6A-6C illustrate partial cross-sectional views of the distal end of the antegrade cardioplegia delivery catheter of FIG. 4A when used to occlude a portion of the ascending aorta.

### DETAILED DESCRIPTION OF EXEMPLARY EMBODIMENTS

Exemplary embodiments of the present disclosure are directed to accessing a body lumen in order to perform a medical or other procedure. For instance, during a minimally invasive surgical procedure, a surgeon may access a body lumen such as the femoral artery or jugular, and extend one or more elements through the vasculature of the patient so as to access a location remote from the access site. Devices that may be extended through the access site and to a remote location of the surgical procedure include catheters, stents, guidewires, other surgical devices, or any combination of the foregoing. Thus, a variety of surgical procedures may be performed within the cavities of the body, particularly including minimally invasive and less invasive surgical procedures in which surgical instruments are introduced through an access site, and thereafter extended through body lumens to a desired location.

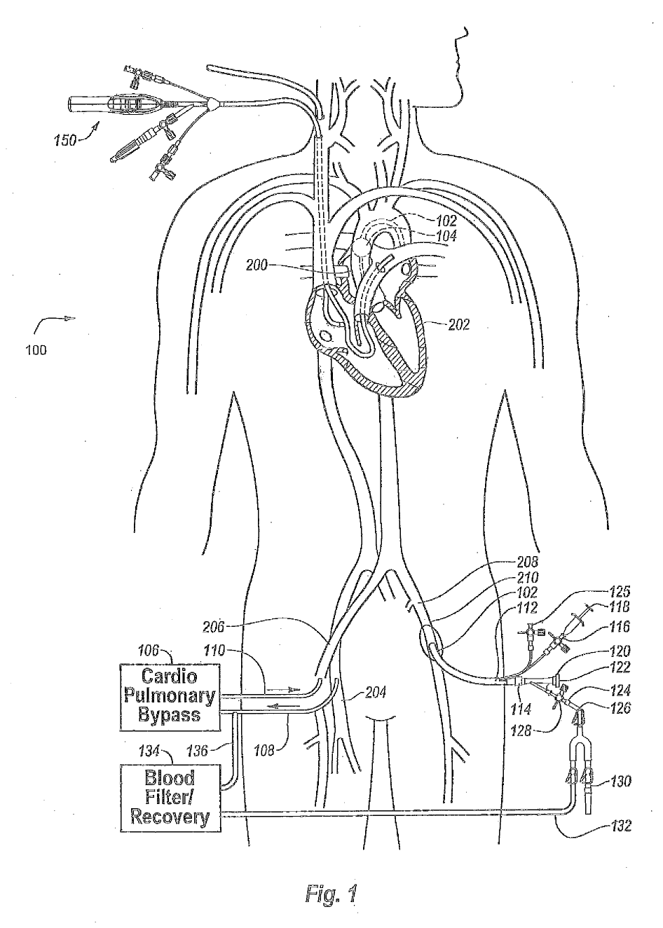

Reference is made to FIG. 1, which schematically illustrates an overall cardiac access system 100 of the present disclosure, as well as various individual components thereof. The cardiac access system 100 may also be referred to as a cardiac occlusion system as, in some embodiments, the cardiac access system 100 may occlude the aorta, coronary sinus, or other cardiac vasculature or lumens. The cardiac access system 100 illustrated in FIG. 1 is intended to provide a general overview of one example of a suitable system for accessing a patient's heart or a portion thereof, and is not intended to be an exhaustive illustration of all components or mechanisms that may be in use during a cardiac procedure.

The cardiac access system 100 may include a first delivery catheter 102. In this embodiment, the first delivery catheter 102 is elongated and is used to access the aorta, although the first delivery catheter 102 may optionally be used to occlude or access other lumens within the body. In the illustrated embodiment, an expandable member 104 located at a distal portion of the delivery catheter 102. When the expandable member 104 is inflated or otherwise expanded, such as is illustrated in FIG. 1, the expandable member 104 may occlude the ascending aorta 200, thereby separating the left ventricle 202 and the upstream portion of the ascending aorta 200 from the rest of the patient's arterial system. Expansion of the expandable member 104 may also be used to securely position the distal end of the delivery catheter 102 within the ascending aorta 200, as the exterior of the expandable member 104 may expand to engage the interior surface of the ascending aorta 200.

In the illustrated embodiment, a cardiopulmonary by-pass system 106 may be used to remove venous blood from the body by, for instance, being placed in fluid communication with the femoral vein 204. A blood withdrawal catheter 108 may connect to the femoral vein 204 and the cardiopulmonary by-pass system 106 and be used to remove blood so as to allow the cardiopulmonary by-pass system 106 to remove carbon dioxide from the blood, oxygenate the blood, and then return the oxygenated blood to the patient. The oxygenated blood may be returned through a return catheter 110 that accesses the femoral artery 206. The oxygenated blood may be returned at sufficient pressure so as to flow throughout the patient's arterial system except for the portion blocked by the expanded occluding member 104 on the aortic occluding catheter 102.

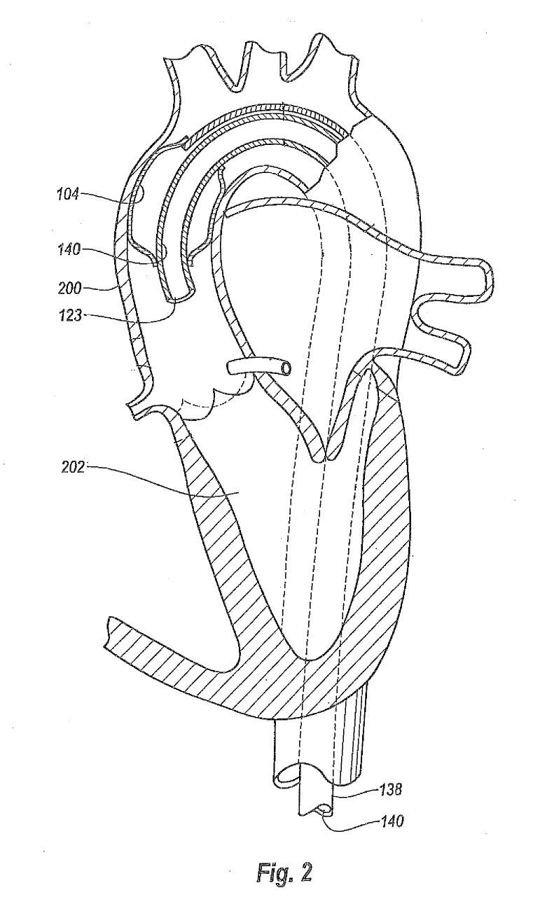

The first delivery catheter 102 of the illustrated embodiment extends through the descending aorta to the left femoral artery 208 and out of the patient through an access site 210, which may be formed as a cut down or in any other suitable manner. A proximal section 112 of the catheter 102 may extend out of the patient through the access site 210. In this embodiment, an adapter 114 may connect to the proximal section 112 of the catheter 102. The adapter 114 is illustrated as having four arms, although a suitable adapter may have more or less than four arms. In one embodiment, a first arm 116 may be adapted for use with the expandable member 104. For instance, an inflation device 118 may be used to inject air or some other fluid that can inflate the expandable member 104. A second arm 120 optionally includes a main access port 122 through which instrumentation or other materials or components may pass. For instance, endovascular devices, valve prostheses, angioscopes, irrigation or cardioplegic fluids, or other components or materials, or any combination of the foregoing, may pass through the main access port 122, through the catheter 102, and out of a distal port 123 (FIG. 2) of the catheter 102. In one example embodiment, the main access port 122 is coupled to a source of cardioplegic fluid (not shown) which is delivered through the catheter 102 to arrest the patient's heart.

The adapter 114 of FIG. 1 also includes a third arm 124 connected to a by-pass line 126 that is provided to direct blood, irrigation fluid, cardioplegia solution, and the like to or from the system. A suitable valve 128 may also be provided to open and close the by-pass line 126 and/or direct fluid passing through the by-pass line to a discharge line 130 or a line 132 to a blood filter and recovery unit 134. A return line 136 may be provided to return any filtered blood to the cardiopulmonary by-pass system 106. In this embodiment, the adapter 114 also includes a fourth arm 125. The fourth arm 125 may, in some embodiments, be in fluid communication with the aortic root (e.g., through a pressure port in the distal end of the catheter 102) and/or a pressure monitoring device (not shown) proximate the adapter 114. Thus, the fourth arm 125 may facilitate measuring of the root pressure within the patient's aorta.

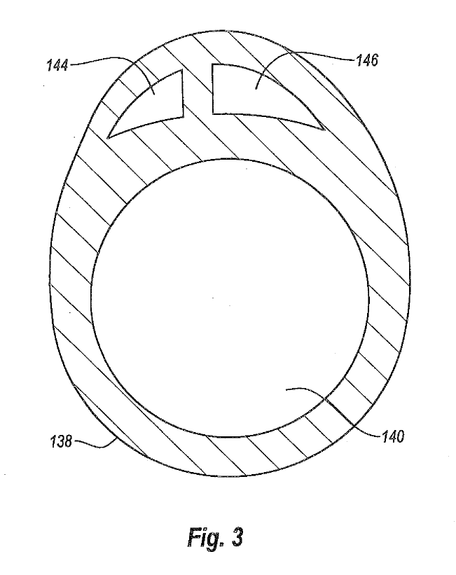

The operation of the first delivery catheter 102 may be understood more completely upon a description of the operation of the catheter 102 within the patient's heart and related vasculature. By way of illustration, and with reference now to FIGS. 2 and 3, the first delivery catheter 102 may include a shaft 138 having an interior lumen 140 accessible through the main access port 122 (see FIG. 1). At the distal end of the shaft 138, there may be a coil 142.

The shaft 138 may include second and/or third interior lumens 144, 146 in some embodiments. The second interior lumen 144 may, for instance, be in fluid communication with the interior of the expandable member 104, which can be an occluding balloon. To inflate the balloon, a gaseous or liquid fluid may pass from the inflation device 118 (FIG. 1) and through the second interior lumen 144 to the expandable member 104. Similarly, a fluid may pass through the second interior lumen 144 and from the expandable member 104, towards the proximal end of the shaft 138 as the expandable member 104 is deflated. The third interior lumen 146 may be in fluid communication with a pressure port at or near the distal end of the shaft 138, as well as with a pressure sensor (e.g., coupled to the fourth arm 125 of the adapter 114 in FIG. 1). Through the third interior lumen 146, pressure in the aortic root and between the aortic annulus and sinotubular junction may be measured.

The shaft 138 and/or expandable member 104 can be shaped and sized in a manner to facilitate insertion and removal of the catheter 102, and or occlusion of the ascending aorta 200 by the expandable member 104. As discussed in greater detail herein, the shaft 138 may have a pre-determined and manufactured contour at the distal end thereof, such that the shaft 138 may have an internal bias causing the shaft 138 to have the desired contour when in an unstressed state. Such contour may generally conform to the shape of all or a portion of the aortic arch and thereby facilitate positioning of the expandable member 104 in the ascending aorta 200, and between the coronary ostia and the brachiocephalic artery. The pre-determined contour at the distal end can be manually or otherwise straightened for introduction of the aortic occlusion catheter 102 in a peripheral artery (e.g., the femoral artery 208 of FIG. 1). Alternatively, the shaft 138 may be manufactured has having a generally straightened configuration, and such shaft 138 may be positioned once inside the patient to obtain the pre-determined contour. Such positioning may occur automatically or may be manually performed by a surgeon. Particular example embodiments of a suitable first delivery catheter 102 are described in greater detail herein.

With reference to FIGS. 1-3, set-up of the cardiac access system 100 will be described in additional detail. More particularly, to set up the cardiac access system 100, the patient may initially be placed under light general anesthesia. The withdrawal catheter 108 and the return catheter 110 of the cardiopulmonary by-pass system 106 can be percutaneously introduced into the right femoral vein 154 and the right femoral artery 156, respectively. An incision 210 may be made in the left groin to expose the left femoral artery 208, and the aortic occluding catheter 102 is inserted into the left femoral artery 208 through the incision 210 and advanced upstream until the expandable member 104 of the occluding catheter 102 is within the ascending aorta 200. Antegrade cardioplegic fluid may then pass through the occluding catheter 102 and into the aorta. In one embodiment, an initial volume of about 1000-1500 ml of cardioplegic fluid is delivered through the interior inner lumen 140 of the aortic occlusion catheter 102, and such delivery may initiate cardioplegic arrest, after which cardioplegic arrest may be maintained by retrograde delivery through the delivery catheter 150.

The operation of the cardiopulmonary by-pass unit 106 can be initiated to withdraw blood from the femoral vein 204 through the catheter 108, remove CO₂ from the withdrawn blood, add oxygen to the withdrawn blood, and then pump the oxygenated blood through the return catheter 110 to the right femoral artery 206. The expandable member 104 may then be inflated or otherwise expanded to occlude the ascending aorta 200, causing the blood pumped out of the left ventricle to flow through a discharge port 123 into the first interior lumen 140 of the occluding catheter 102. The blood may flow through the interior lumen 140 and out the third arm 124 of the adapter 114 into the by-pass line 126 and then into a blood filter and recovery unit 134 through the valve 128 and line 132. For blood and irrigation fluids containing debris and the like, the position of the valve 128 may be changed to direct the fluid through the discharge line 130.

With the cardiopulmonary by-pass system in operation, the heart may become completely paralyzed and stop pumping. The left atrium may become decompressed and the ascending aorta can be blocked by the expandable member 104 on the occluding catheter 102. At such point in time, the heart may be appropriately prepared for a cardiac procedure. The procedures with which the system and method of the present disclosure are useful include thoracoscopic coronary artery bypass grafting, thoracoscopic or endovascular repair or replacement of the mitral, aortic and other valves, thoracoscopic repair of atrial or ventricular septal defects and other congenital defects, transmyocardial laser revascularization, electrophysiological mapping and ablation, and various other procedures which require or would benefit from the inducement of cardioplegic arrest. The present disclosure may also be utilized to induce cardioplegic arrest in conventional open surgical procedures as a substitute for conventional external aortic cross-clamps and conventional cardioplegia cannula introduced directly into the heart and/or aorta.

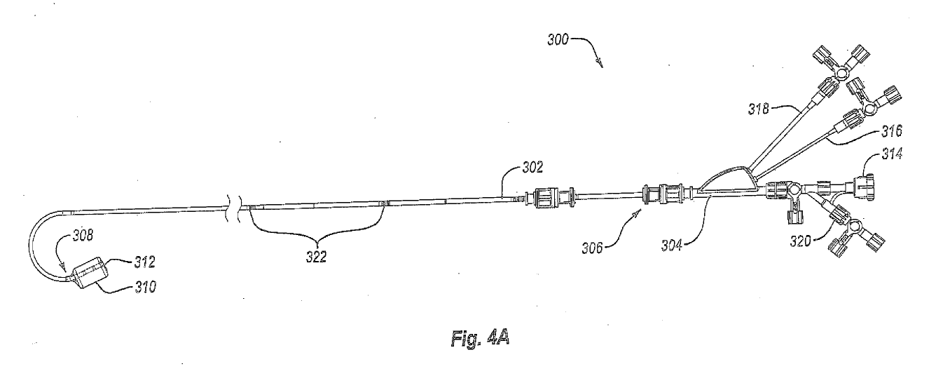

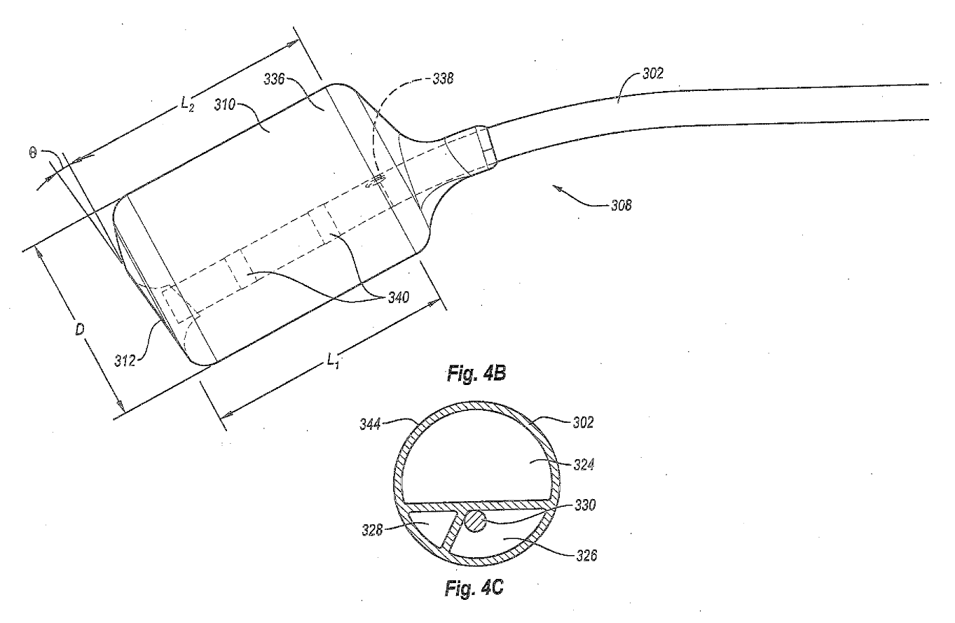

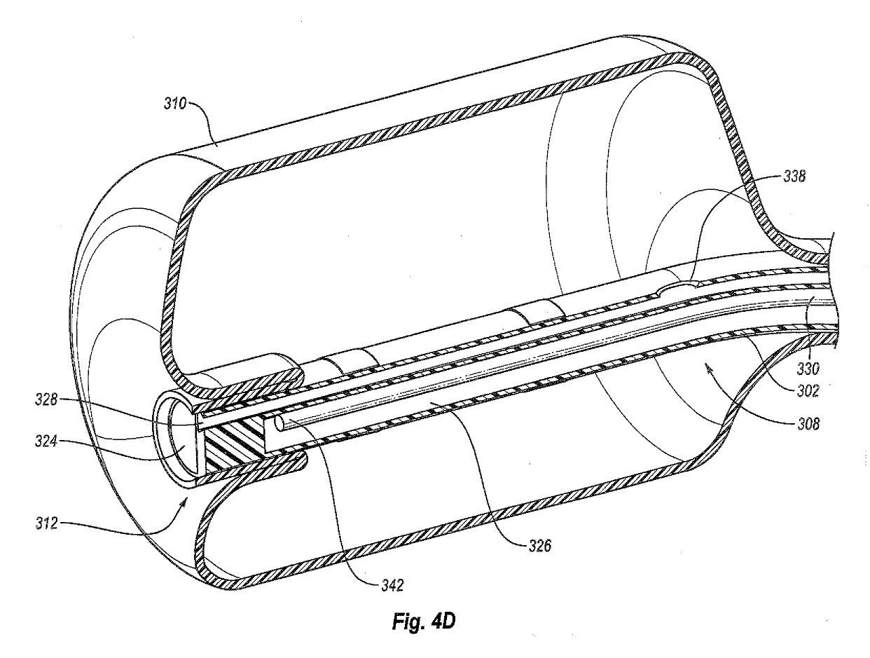

Turning now to FIG. 4A, an exemplary embodiment of a delivery catheter 300, and more particularly an antegrade cardioplegia delivery catheter or aortic occlusion catheter, is illustrated and described in additional detail. In the illustrated embodiment, the delivery catheter 300 includes a catheter shaft 302 that can be inserted into a patient and located at a desired location—such as within the ascending aorta of the patient. Accordingly, the shaft 302 may have a length such that when a distal end 308 of the shaft 302, including an expandable member 310, is at a desired location within the patient, a proximal end 306 of the shaft 302 may remain exterior to the patient. The proximal end 306 of the shaft 302 may be positioned, for instance, adjacent a peripheral access site, such as in the femoral artery to facilitate a minimally invasive procedure. A hub 304 may also be attached to the shaft 302. The hub 304 may serve any number of purposes. For instance, in the embodiment shown in FIG. 4A, the hub 304 may have various extension arms 314, 316, 318, 320 that serve various purposes. Such extension arms may, for instance, facilitate expansion of the expandable member 310, delivery of cardioplegic fluid, monitoring of pressure or characteristics within the vasculature at the distal tip 312, insertion of guidewires, stents, replacement valves, other devices or components, or any combination of the foregoing.

Similar to the mariner discussed above with regard to the system 100 of FIGS. 1-3, and more particularly with respect to the delivery device 102 of FIGS. 1-3, the delivery catheter 300 may be used to occlude a portion of a patient's vasculature at or near the heart, while also supplying cardioplegic fluid to the heart. An exemplary manner in which the delivery catheter 300 can be used to occlude vasculature is may be understood particularly with reference to FIG. 4B-4D.

In particular, to facilitate such occlusive functions, the delivery catheter 300 may include an expandable member 310. The expandable member 310 may be generally positioned at the distal end 308 of the shaft 302, and may be proximate or adjacent a distal tip 312 of the shaft 302. The expandable member 310 may be configured to vary its size, diameter, or other dimension in any suitable manner. In some embodiments, the expandable member 310 is an expandable balloon. By way of illustration, the expandable member 310 may be formed of a flexible material. The expandable member 310 may, for instance, be polyurethane, PTFE, or other material that is blow-molded, dip-molded, or otherwise formed. The expandable member 310 may also be formed of other materials, formed in other manners, or take other forms. For instance, the expandable member 310 need not be a balloon, and could be any other suitable type of selectively expandable element.

The expandable member 310 of FIGS. 4B and 4D is illustrated in an expanded state. It should be appreciated, however, that the expandable member 310 may be inserted into a patient while in a collapsed, partially collapsed, or other state that may allow the expandable member 310 to pass more easily through the patient's vasculature. In some embodiments, as the expandable member 310 moves through the vasculature, the expandable member 310 does not substantially occlude the vasculature, at least not until the distal tip 312 of the shaft 302 is at or near an intended location. Once at the desired location, the expandable member 310 may be expanded.

Expansion of the expandable member 310 may be performed in any suitable manner. For instance, where the expandable member 310 is an expandable balloon, a fluid may be selectively passed through the shaft 302 and into the expandable member 310. In FIG. 4A, for instance, the shaft 302 may connect to the hub 304, and may be in fluid communication with one or more of the various extension arms 314, 316, 318, 320. The shaft 302 may have one or more lumens therein to receive fluid, instruments, or other items. For instance as best shown in FIG. 4C and 4D, the shaft 302 may have a multi-lumen design. Each of the multiple lumens 324, 326, 328 may be in communication with the one or more extension arms 314, 316, 318, 320 (FIG. 4A) which may act as access ports to the respective lumens 324, 326, 328.

In the illustrated embodiment, the shaft 302 may include a primary lumen 324 and multiple secondary lumens 326, 328. The secondary lumen 326 may, for instance, extend along a length of the shaft 302 and terminate at a location within the expandable member 310. As shown in FIG. 4D, for instance, the secondary lumen 326 may terminate near the distal tip 312 of the shaft 302. The secondary lumen 326 may be in fluid communication with an inflation port 338 that extends through a sidewall of the shaft 302. The inflation port 338 can be within the expandable member 310, such that as fluid is inserted through the lumen 326 and exits the shaft 302 through the inflation port 338, the expandable member 310 may inflate or otherwise expand. Conversely, fluid dispelled from the expandable member 310 may pass through the inflation port 338 and into the shaft 302 as the expandable member 310 contracts.

The expandable member 338 may have any number of suitable constructions or configurations. For instance, in FIG. 4B, the expandable member 310 is illustrated as an inflated balloon having a generally elongated, hexagonal side profile, and with the shaft 302 being eccentric relative to the central axis 332 of the expandable member 310. The particular dimensions and configuration of the expandable member 310 can vary as desired so as to, for example, occlude an ascending aorta of a patient. In some embodiments, the dimension D corresponds to a diameter or width of the expandable member 310 and can generally correspond to the width of the ascending aorta at a desired occlusion location. For instance, the ascending aorta in an average adult may measure between about 3.5 and about 3.8 cm. Accordingly, in some embodiments, the expanded diameter D is about 3 cm to about 4 cm. The working length L₁ may also correspond to a length of the expandable member 310 that can engage the lower surface of the aorta to facilitate occlusion. In general, an increased working length L₁ increases the surface area for contact with the aorta and favors stabilizing the position of the expandable.

The expandable member 310 may generally be considered has being divided by the shaft 302 into a lower portion 334 and an upper portion 336. In this embodiment, the upper and lower portions 334, 336 have different lengths L₁, L₂. The eccentric profile of the shaft 302 may provide differing sizes of portions 334, 336; however, the general shape of the expandable member 310 may additionally or alternatively be varied. In this embodiment, for instance, the upper portion 336 may have a side surface extending at an angle from the distal tip 312, and to an upper contact surface, such that the length of the upper contact surface has the length L₂. The length L₂ may be greater or smaller than the working length L₁. In the illustrated embodiment, for instance, the angle φ may be between about ten and about twenty-five degrees, and more particularly between about thirteen and about twenty-one degrees. For instance, the angle φ may be between about fifteen and about eighteen degrees, such that the length L₁ is greater than the length L₂.

It should be appreciated in view of the disclosure herein that the expandable member 310 is but one example of a suitable expandable member, and that other expandable members may be used. For instance, in other embodiments, the expandable member 310 may be spherical, trapezoidal, cylindrical, barrel-shaped, or otherwise configured. Moreover, the degree of eccentricity of the shaft 302 relative to the central axis 332 may also be varied. For instance, the shaft 302 may be concentric with the axis 332 (i.e., 0% eccentricity) or may vary up to nearly 100% eccentricity (i.e., the shaft at the upper or lower surface of the expandable member). In one embodiment, the eccentricity of the shaft 302 may be between about 5% and about 36%.

To facilitate cardioplegic functions of the delivery catheter 300, the delivery catheter 300 may allow cardioplegic fluid to be passed from a fluid source or reservoir and into the ascending aorta or other location within a patient. FIGS. 4C and 4D illustrate a particular manner in which such features can be provided. For instance, as noted previously, the shaft 302 optionally includes multiple fluid conduits, channels, lumens, or other features. In particular, in the illustrated embodiment, the shaft 302 may include a primary lumen 324 that is optionally in fluid communication with an extension arm 314 that acts as a port to allow the introduction of cardioplegic fluid, guidewires, surgical instruments, or other elements. Cardioplegic fluid may be pressurized and passed through the lumen 324 towards the distal tip 312 of the shaft 302. As best shown in FIG. 4D, the distal end of the shaft 302 may include an opening generally corresponding to the lumen 324. For instance, the lumen 324 may be open at the distal tip 312 such that the pressurized cardioplegic fluid exits the shaft 302 distal to the expandable member 310.

In some embodiments, the distal tip 312 may be integrally formed with the shaft 302, although in other embodiments the distal tip 312 and shaft 302 are separate components that are bonded together. For instance, the distal tip 312 may be a molded, extruded, or otherwise formed component that is bonded to the shaft 302 using a thermal, adhesive, laser welding, overmolding, or other procedure. As best shown in FIGS. 4B and 4D, in some embodiments, the expandable member 510 may extend at least slightly distal relative to the distal tip 512. In such an embodiment, the lumens 524, 528 at the distal tip 512 may be protected by the expandable member 510. For instance, a distal leg of the expandable member 510 may connect to the distal tip 512 of the shaft 502. If the distal tip 512 is lodged into a vascular wall, the lumens 524, 528 may remain patent and able to deliver fluid, monitor cardiac or vascular characteristics, receive fluid, or the like.

The delivery catheter 310 may provide still other features and uses. For instance, in accordance with another embodiment, cardiac and/or vascular characteristics can be monitored using the delivery catheter 310. Such characteristics may include, for instance, flow rates, beat rates (if any), pressure, or dimensions, or other characteristics. In one embodiment, such as where the delivery catheter 310 is configured to occlude the ascending aorta, the delivery catheter 310 may be adapted to measure a pressure within the aorta, such as the aortic root pressure. As shown in FIGS. 4C and 4D, for instance, a secondary lumen 328 may extend to a vent at or near the distal tip 312 of the shaft 302. The secondary lumen 328 may be in fluid communication with a pressure monitoring device (e.g., through a connection at extension arm 318 of FIG. 4A), thereby allowing root aortic pressure to be monitored throughout a surgical or other procedure.

As will be appreciated in view of the disclosure herein, the delivery catheter 300 may thus be configured to provide any number of features. In accordance with some embodiments, the shaft 302 may be adapted to provide still other features and aspects. For instance, as shown in FIG. 4A, the shaft 302 may include one or more markings 322 thereon. Such markings may be bands, ink, radiopaque markers, or otherwise structured to facilitate visualization inside or outside the patient. For instance, in one embodiment, the markings 322 are radiopaque markings that are visible under transesophageal echocardiography visualization or other visualization techniques, so as to facilitate positioning of the shaft within a patient. Where an expandable member 310 is to be placed at a particular location, the expandable member 310 may optionally include additional markings (e.g., platinum iridium markers) to facilitate visualization. For instance, as best shown in FIG. 4B, one or more markings 340 may be placed on, within, or proximate the expandable member 310 to thereby allow identification of a position of the expandable member 310 when a particular visualization technique is used.

The shaft 302 may be otherwise structured to facilitate insertion, removal, and/or placement of the delivery catheter 300 during a surgical procedure. For instance, as shown in FIGS. 4C and 4D, the shaft 302 may include two components. Such components include, in this embodiment, a body element 344 and a core element 330. The body element 344 may, for instance, generally define the shape of the shaft 302 and the lumens 324, 326, 328 within the shaft. In one embodiment, the body element 344 may be formed of any suitable material and using any number of different manufacturing processes. For instance, the body element 344 may be formed from a flexible material that can bend as the shaft 318 translates through a patient's vasculature, to thereby match contours within the patient's body. Suitable materials may include, for instance, ethylene tetrafluoroethylene (ETFE) or polytetraflourothylene (PTFE). In another embodiment, the outer shell 305 is formed from a biocompatible material such as Pebax®. A body element 344 is produced from Pebax® which may be extruded and can even be extruded to simultaneously define multiple lumens. Accordingly, the body element 344 is optionally a multi-lumen extrusion, although in other embodiments the body element 344 may be formed as a separate fluid lines layered together with a heat shrink wrap holding them together. In at least some embodiments, the durometer of the body element 344 may be between about 20 to about 80 Shore D. Such durometer may also change along the length of the body element 344. For instance, the durometer of the distal tip 312 may be lower relative to the durometer at a proximal end of the shaft 302.

In at least some embodiments, the body element 344 is a solid extrusion, rather than a wrap that includes coils or a supporting exoskeleton, wire frame, or the like. In at least one embodiment, such as that shown in FIGS. 4C and 4D, the shaft 302 may include a core 330 within the secondary lumen 326. The secondary lumen 326 may, as discussed previously, be used for facilitating expansion of the expandable member 310, or for any other desired feature. According to one aspect, the core 330 may be a wire extending along all or a portion of the length of the shaft 302. The core 330 may have a stiffness and strength that provides additional column stiffness to facilitate placement of the shaft 302. The core 330 may additionally, or alternatively, provide kink resistance or define a desired shape of the shaft 302.

For instance, as reflected in FIG. 4A, the distal end 308 of the shaft 302 may have a bend, curve, or other shape. In some embodiments, the shaft 302 may be configured to pass through the descending aorta and into the ascending aorta. To do so, the curved distal end 308 may pass around a relatively tight curve radius, namely the curve radius defined by the aortic arch.

While curvature of the distal end 308 may be produced by allowing the body element 344 and/or core 330 to be made of a flexible material, in other embodiments the core is pre-designed and manufactured to maintain a specific curved profile. In still other embodiments, such curved profile may be selectively activated in the shaft 302. To obtain these and other characteristics, in one embodiment, the core 330 can be comprised of biocompatible materials that are at least temporarily deformable. Suitable biocompatible materials include, for example, superelastic and/or shame memory materials (e.g., copper-zinc-aluminum; copper-aluminum-nickel; nickel-titanium alloys known as Nitinol; cobalt-chromium-nickel alloys, cobalt-chromium-nickel-molybdenum alloys, nickel-titanium-chromium alloys, and the like). In addition, and by way of representation only, other suitable materials may include stainless steel, silver, platinum, tantalum, palladium, cobalt-chromium alloys, niobium, iridium, any equivalents thereof, alloys thereof or combinations thereof.

Where the core 330 is formed of a shape memory material, the core 330 can be shaped in a manner that allows deformation from a pre-determined curved memory shape while the core 330 is outside the body lumen of a patient, but which can automatically retain the curved memory shape while within a body lumen. Shape memory materials have a shape memory effect in which they can be made to remember a particular shape. Once a shape has been remembered, the shape memory material may be bent out of shape or deformed and then returned to its original shape by unloading from strain or by heating. In one embodiment, for instance, the core 330 is formed of a shape memory material manufactured to remember, over at least a portion of the core 330, a curved shape generally corresponding to the curvature of an aortic arch. Such curvature need not correspond directly to the aortic arch, or may generally correspond to any of various portions of the aortic arch. For instance, the curvature of the core 330 may correspond to an upper curvature or central curvature of the aortic arch. As discussed in greater detail herein, the core 330 may alternatively have a memory shape configured to correspond to the bottom curvature of the aortic arch.

Activation of the core 330 to transition from a deformed state to a remembered shape may be performed in any manner, such as by applying a force on the core 330 (e.g., to induce a strain), or by placing the core 330 at a desired temperature. For instance, in one embodiment, the core 330 is trained to be thermally activated and transition from a deformed shape to a pre-determined shape when the core 330 is placed at about body temperature (e.g., about 37° C.). As the core 330 may be placed within the body element 344, the change in shape of the core 330 may also cause the body element 340 to change shape, thereby changing the shape and profile of the distal end 308 of the shaft 302. At body temperature, or when activated in another mariner, the core 330 may move to the trained shape such that the radial strength increases, whereas at room temperature or another non-activated state, the core 330 may be relatively weak in the radial direction and may be readily deformable.

In accordance with some embodiments, the core 330 may be a wire, although the core 330 may take other forms. As best illustrated in FIG. 4D, the core 330 may be a wire having a variable cross-sectional shape. In particular, in at least one embodiment, the core 330 may have a distal end 342 at least proximate the distal end 308 of the shaft 302. As the core 330 approaches the distal tip 312 of the shaft 302, the size of the core 330 may, in some embodiments, decrease, such as by having a tapered, stepped, or other configuration. In such a manner, the strength of the core 330 at the distal tip 312 may be decreased, thereby also reducing the force that the core 330 can exert at the distal tip 312. With reduced force at the distal tip 312, trauma to a patient's vasculature may be decreased.

The shaft 302 and the hub 304 may be formed in any number of manners, or have any other number of features or configurations. For instance, the size of the shaft 302 may be varied as desired. In accordance with one embodiment, the shaft 302 may have an outer diameter of between about eight and ten French, so as to be passable from a peripheral artery through the descending aorta, and into the ascending aorta as described herein. Depending on other uses of the delivery catheter 300, the patient with whom the catheter 300 is used, or other factors, the size of the shaft 302 may be larger than ten French, or smaller than eight French.

The shaft 302 may be connected to the hub 304 in any suitable manner. For instance, in one embodiment, the shaft 302 and the hub 304 are an integral unit and are molded together. In another embodiment, the shaft 302 may be formed separate from the hub 304 and thereafter attached to the hub. For instance, the shaft 302 may be extruded and the hub 304 may be molded and then bonded to the shaft 302. Such bonding may be performed by a thermal bonding, overmolding, adhesive, or other attachment procedure. The extension arms 314, 316, 318, 320 may be similarly formed. For instance the extension arms 314, 316, 318, 320 may be molded and integrally formed with the hub 304. In some embodiments, the extension arms 314, 316, 318, 320 are flexible, but may be rigid. In at least one embodiment, some extension arms (e.g., arms 316, 318) may be flexible while other extension arms (e.g., arms 314, 320) are substantially rigid. As discussed herein, the extension arms 314, 316, 318, 320 may serve as ports and facilitate balloon inflation, aortic root pressure monitoring, cardioplegia delivery, aortic root venting, or other aspects.

In at least one embodiment, the hub 304 may further facilitate proper positioning of the distal end 308 of the shaft 302 within a patient. For instance, as discussed previously, the shaft 302 may have a predetermined curve or other profile. The predetermined curve or other profile may be fixed in relation to the orientation of the hub 304. Indicia (not shown) may be placed on the hub 304 to indicate the direction of the curved profile of the shaft 302 such that once the distal end 308 of the shaft 302 is within a patient, the surgeon or other operator will be aware by glancing at the hub 304 as to what direction the shaft 302 will bend or curve. In other embodiments, the hub 304 may be asymmetric. A direction of asymmetry may correspond with the curve of the shaft 302, thereby allowing the surgeon to glance at the hub 304, view the asymmetry, and know which direction the shaft 302 curves.

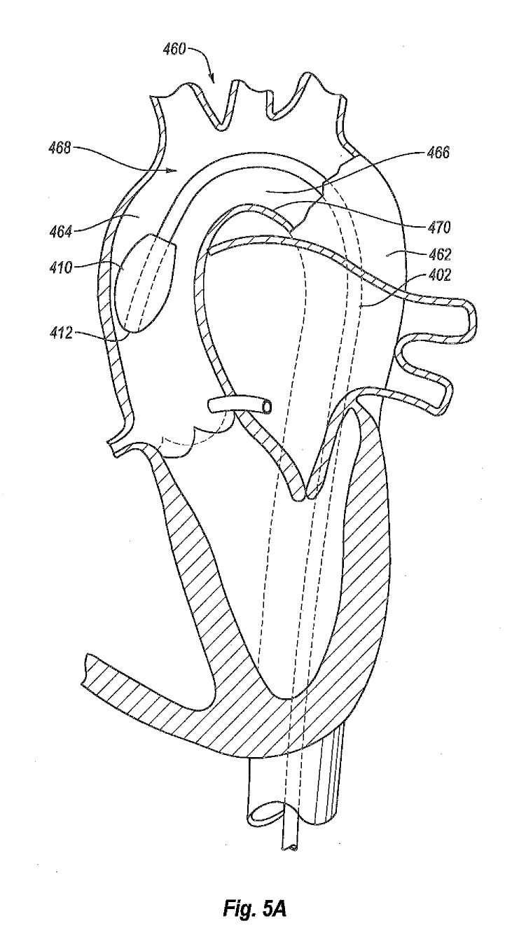

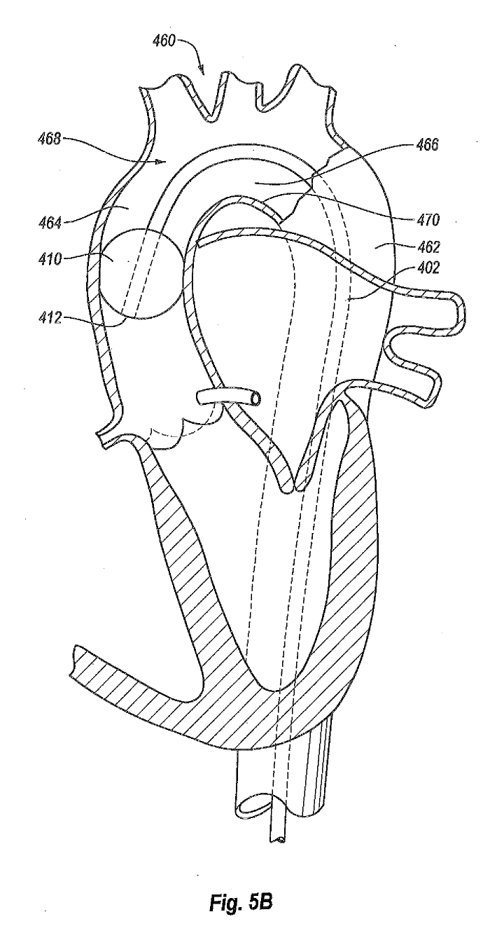

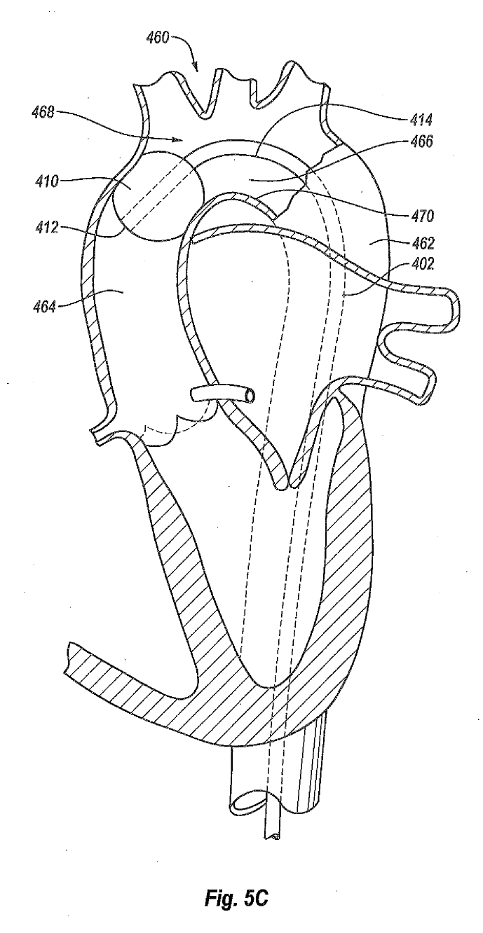

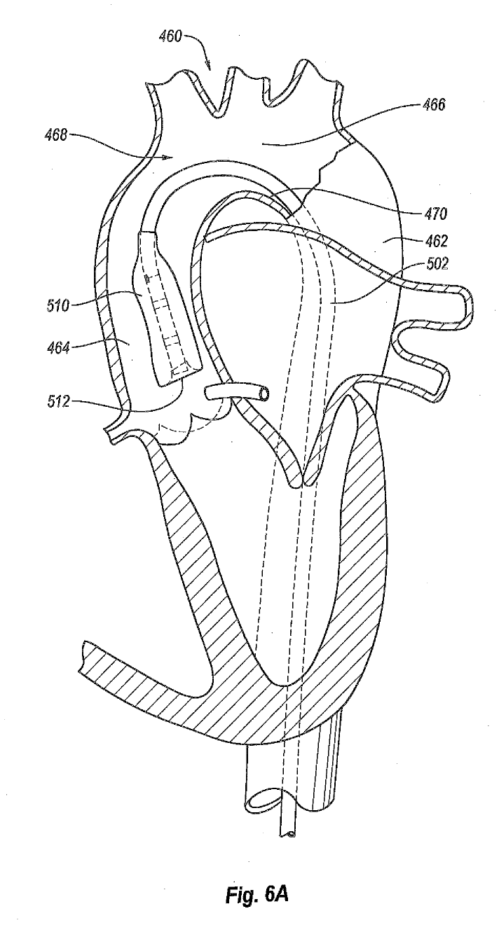

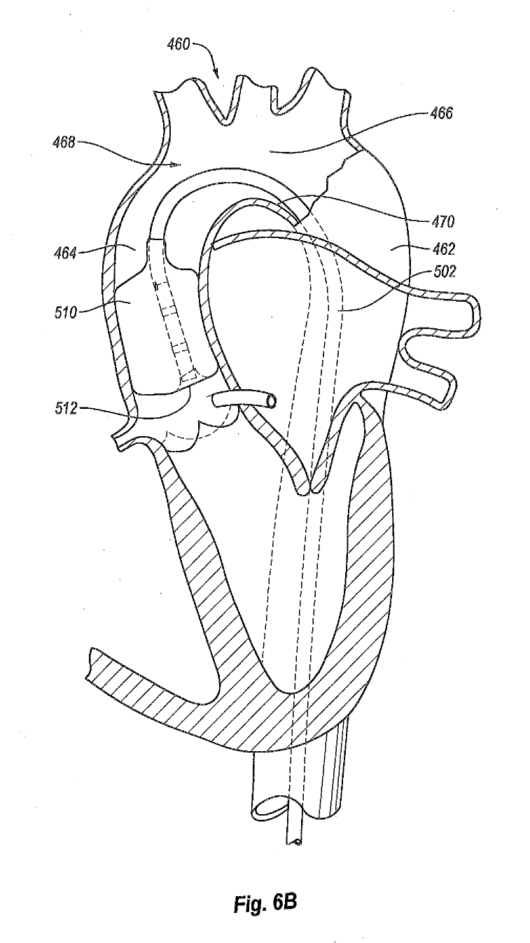

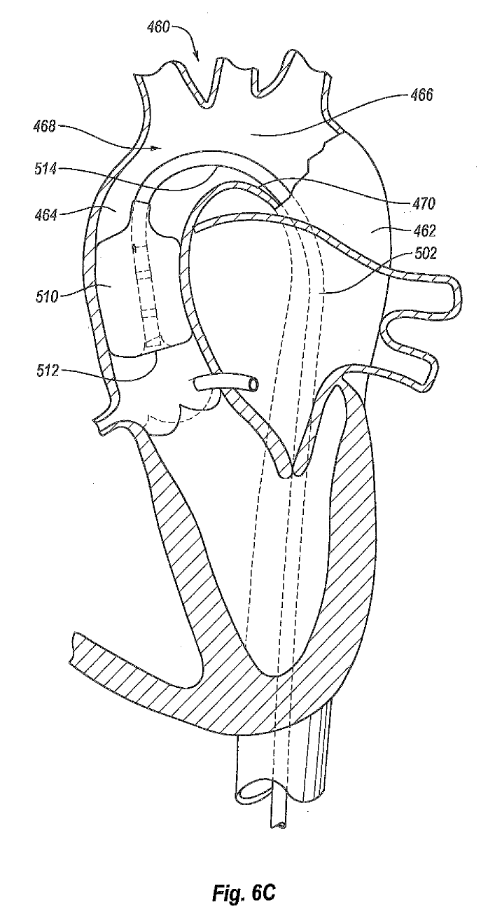

Turning now to FIGS. 5A-5C and FIGS. 6A-6D, various exemplary aspects of embodiments of the present disclosure are illustrated and described in greater detail, particularly with regard to the manner of use of an antegrade cardioplegia delivery catheter that occludes the ascending aorta of a patient. For instance, FIGS. 5A-5C generally illustrate a process of inserting a shaft 402 and expandable member 410 of a delivery catheter into a patient's aorta 460, expanding the expandable member 410, and retracting expandable member 410 to secure the expandable member in an occluding position.

More particularly, in FIG. 5A, a shaft 402 and expandable member 410 may be passed through the descending aorta 462, around the aortic arch 466, and into the ascending aorta 464. During such movement, the expandable member 410 may be in a deflated or otherwise contracted state. In order to facilitate placement of the expandable member 410 and a distal tip 412 within the ascending aorta 464, the shaft 402 may be flexible. In particular, the shaft 402 may bend to generally correspond to a curve of the aortic arch 466. For instance, the aortic arch 466 may have an upper profile 468 and a lower profile 470. The shaft 402 may bend so as to generally have a curve that extends partially between the upper and lower profiles 468, 470 of the aortic arch 466.

The expandable member 410 and distal tip 412 may be located using any suitable visualization technique. Once positioned in the desired location, the expandable member 410 may be expanded using any suitable manner, including those described herein. For instance, the expandable member 410 may be a balloon that is inflated to substantially occlude the ascending aorta 464. In FIG. 5B, for instance, the expandable member 410 has a generally spherical shape and the shaft 402 is generally concentric within the expandable member 410.

Inflation of the expandable member 410 on the distal end of the shaft 402 can fix the distal tip 412 of the shaft 402 within the ascending aorta 464 and isolate the left ventricle of the heart and the upstream portion of the ascending aorta 464 from the rest of the arterial system downstream from the expandable member 410. The passage of any debris or emboli, solid or gaseous, generated during a cardiovascular procedure to regions downstream from the site can be substantially prevented by the expanded expandable member 410. Fluid containing debris or emboli can be removed from the region between the aortic valve and the occluding expandable member 410 through an interior lumen of the shaft 402. A clear, compatible fluid (e.g., an aqueous based fluid such as saline) delivered through an interior lumen or the cardioplegic fluid may be maintained in the region wherein the cardiovascular procedure is to be performed to facilitate use of an angioscope or other imaging means that allows for direct observation. Such use of a delivery catheter may be particularly useful in the removal of an aortic heart valve and replacement thereof with a prosthetic heart valve which procedure is described in U.S. Pat. No. 5,738,652, which patent is hereby expressly incorporated herein by this reference, in its entirety.

The expandable member 410 may have forces applied thereto that cause the expandable member 410 to shift position. For instance, as cardioplegic fluid is expelled from the distal tip 412, the fluid flow may generally cause the expandable member 410 to move upward through the ascending aorta 464 and towards the aortic arch 466. Other forces may also be applied, for instance, a decrease in perfusion pressure may also cause the expandable member 410 to move towards the aortic arch 466. In contrast, the systemic blood pressure, increases in root vent suction, or increases in perfusion pressure may tend to cause the expandable member 410 to move further into the ascending aorta 464 and away from the aortic arch 460.

Migration of the expandable member 410 may be particularly likely where slack is present in the shaft 402. Accordingly, to minimize migration of the expandable member 410, a surgeon may pull on the delivery catheter so as to at least partially retract the shaft 402. For instance, a surgeon may pull two to three inches of slack out of the shaft 302. As a result, the expandable member 410 may move towards the aortic arch 466. In retracting the expandable member 410, external surfaces of the expandable member 410 may also more fully engage the upper and lower portions of the ascending aorta 464, thereby more securely positioning the expandable member 410 as it occludes the aorta.

As shown in FIG. 5C, the shaft 402 may have a curved profile 414 that generally corresponds to a portion of the aortic arch 466. In this embodiment, for instance, the curved profile 414 allows the shaft 402 to curve around the aortic arch 466 generally between the upper profile 468 and lower profile 470 of the aortic arch 466. The shaft 402 may be generally mid-way between the upper and lower profiles 468, 470, although such is not necessary. For instance, the shaft 402 may be generally flexible such that the profile 414 adapts to a suitable geometry that allows the expandable member 410 to remain at the illustrated occluding position.

When the slack is pulled from the shaft 402, such that the expandable member 410 is secured within the ascending aorta 464, the distal tip 412 of the shaft 402 may migrate and change orientation within the ascending aorta 464. More particularly, in the illustrated embodiment, the distal tip 412 may be positioned at an angle relative to the ascending aorta 464. As noted herein, cardioplegic fluid may, in some instances, be perfused to the ascending aorta 464 through the distal tip 412. Where the distal tip 412 is angled, pressurized fluid may exit the distal tip 412 and be directed at the upper wall of the ascending aorta 464. In some cases, such delivery may be undesirable as the pressurized fluid may cause trauma or other damage to the interior wall of the ascending aorta 464. Generally speaking, the shape of the expandable member 410, curvature of the shaft 402, and location of the shaft 402 within the expandable member 410 may each contribute to the orientation of the distal tip 412.

FIGS. 6A-6C illustrate an alternative embodiment of a delivery catheter that may be used to occlude the aorta 460 and deliver cardioplegic fluid to the ascending aorta 464. In particular, FIG. 6A illustrates an embodiment similar to that shown in FIG. 5A. More particularly, a shaft 502 is connected to an expandable member 510 and passed through the descending aorta 462, around the aortic arch 466, and into the ascending aorta 464 while the expandable member 510 is in a deflated or other contracted state.

As shown in FIG. 6A, the distal end of the expandable member 510 envelops the distal tip 512. This distal tip 512 envelopment by the less rigid material of the expandable member 510 reduces the risk of perforation of the vasculature as the delivery catheter is tracked through the femoral artery into the aorta. Once within the ascending aorta 464, the expandable member 510 may be inflated or otherwise expanded as shown in FIG. 6B. In the illustrated embodiment, the expandable member 510 has an elongated construction. The particular shape of the expandable member 510 may vary. For instance, the expandable member 510 may be hexagonal, trapezoidal, cylindrical, barrel-shaped, or have another suitable configuration.

As further shown in the illustrated embodiment, the shaft 502 may be eccentrically positioned relative to the expandable member 510. As a result, an upper portion of the expandable member 510 may be larger in at least one dimension that a lower portion of the expandable member 510. The upper portion of the expandable member 510 may be adapted to engage the upper surface 468 of the aorta 460, while the lower portion of the expandable member 510 may be adapted to engage the lower, or bottom surface 470 of the aorta 460. By engaging the upper and lower surfaces 568, 470 of the aorta 460, the expandable member 510 may substantially occlude the aorta 460.

In some embodiments, the expandable member 510 may also be retracted so as to secure the expandable member 510 in a position that reduces migration of the expandable member 510. As shown in FIG. 6C, the shaft 502 may be at least partially retracted so as to move the expandable member 510 within the ascending aorta 464 and towards the aortic arch 466. In some embodiments, retracting the shaft 502 may require only a minor amount of slack to be removed from the shaft 502. For instance, in the illustrated embodiment, the shaft 502 has a curved profile 514 generally corresponding to the contour of the aortic arch 466. In at least one embodiment, the curved profile 514 is specifically configured to generally correspond to the size and contour of the lower or bottom surface 470 of the aortic arch 466. Consequently, as the shaft 502 is inserted into the aorta 460, the shaft 502 tracks along the bottom surface 470; this can minimize the travel distance of the shaft 502. With reduced travel distance, there may be less slack in the shaft 502. Moreover, as the shaft 502 can mirror the contour of the bottom surface 470, retraction of the shaft 502 also causes the shaft 502 to track along the bottom surface 470 of the aorta 460. The retraction distance, and thus the amount of slack that is pulled out, may thus be reduced. For instance, less than two inches, and potentially less than one inch of slack may be removed in order to securely position the expandable member 510 in a desired position. For instance, in some embodiments, about three centimeters of slack may be removed from the shaft 502.

As further shown in FIG. 6C, once the slack has been pulled out and the expandable member 510 secured in an occluding position within the ascending aorta 464, the distal tip 512 may remain positioned within the ascending aorta 464. In this embodiment, the distal tip 512 is generally oriented to be about parallel with the ascending aorta 464. Consequently, in embodiments in which cardioplegic fluid is passed out of the distal tip 512 and perfused to the ascending aorta 464, the cardioplegic fluid may be directed along the pathway of the ascending aorta 464, rather than into a sidewall of the ascending aorta 464. The substantially parallel alignment of the distal tip 512 may thus reduce a risk of trauma to the ascending aorta 464 during a surgical procedure.

The positioning of the distal tip 412 in a parallel or substantially parallel position within the ascending aorta 464 may result from a combination of one or more factors, including shape of the expandable member 510, eccentric positioning of the shaft 502 relative to the expandable member 510, the curve profile 514 of the shaft 502, material properties of the shaft 502, or other factors, or any combination of the foregoing. For instance, in one embodiment, the expandable member 510, eccentric position of the shaft 502, and material properties of the shaft 502 may be similar to those described above with respect to delivery catheter 300 of FIGS. 4A-4D. The curve profile 514 of the shaft 502 may also be similar to those previously described. By way of illustration, in some embodiments, the shaft 502 may include a core (not shown) that is formed at least partially of a memory material that has a pre-determined and manufactured curve profile. Such profile may vary as desired based on the patient, size of the aorta, or other factors. In one example embodiment, the curve profile 514 is configured to have a radius of curvature of between about ten and about twenty five millimeters. More particularly, in some embodiments, the radius of curvature at the curve profile 514 of the shaft 502 may be between about fifteen and about twenty one millimeters. In a still more particular embodiment, the radius of curvature at the curve profile 514 of the shaft 502 may be between about seventeen and about nineteen millimeters.

Unless described otherwise, the various components of the systems and devices of the present disclosure can be formed of conventional materials using conventional manufacturing techniques. The dimensions of the various components are selected so that they perform their intended functions in their intended environment, but are not intended to limit the scope of the present disclosure unless expressly claimed.

Although the foregoing invention has been described in some detail by way of illustration and example, for purposes of clarity and understanding, certain changes and modifications will be obvious to those with skill in the art in view of the disclosure herein. The described embodiments are to be considered in all respects only as illustrative and not restrictive. Thus, all changes which come within the meaning and range of equivalency of the claims are to be embraced within their scope.

What is claimed is:

1. A device for occluding a patient's ascending aorta, comprising:
a hub having one or more ports;
an elongated body having opposing proximal and distal ends, the elongated body defining one or more lumens extending at least partially between the proximal and distal ends of the elongated body, and each of the one or more lumens being in fluid communication with at least one of the one or more ports of the hub, wherein the elongated body has a pre-configured, selectively actuated curve profile at least proximate the distal end of the elongated body; and
an expandable member at the distal end of the elongated body, the expandable member being selectively changeable between expanded and contracted states.

2. The device recited in claim 1, wherein the elongated body is a shaft.

3. The device recited in claim 1, wherein the elongated body is a multi-lumen extrusion.

4. The device recited in claim 3, wherein each of a plurality of lumens of the multi-lumen extrusion is in fluid with at least one of the one or more ports, the plurality of lumens collectively being configured for:
inflation of the expandable member;
cardioplegic fluid delivery;
cardiac characteristic monitoring; and
root venting.

5. The device recited in claim 1, wherein the elongated body has a distal tip that is enveloped by the expandable member while the expandable member is in a contracted state.

6. The device recited in claim 1, wherein the elongated body is not reinforced by a wire mesh or frame.

7. The device recited in claim 1, further comprising a core extending within one of the one or more lumens of the elongated body.

8. The device recited in claim 7, wherein the core is a wire having a tapered distal end.

9. The device recited in claim 7, wherein the core substantially defines the pre-configured, selectively actuated curve profile of the elongated body.

10. The device recited in claim 7, wherein the core is formed of a memory material, the memory material defining the pre-configured, selectively actuated curve profile of the elongated body.

11. The device recited in claim 10, wherein the memory material is selectively actuated at one or more temperatures.

12. The device recited in claim 11, wherein the memory material is selectively actuated at or near body temperature.

13. The device recited in claim 1, wherein the pre-configured, selectively actuated curve profile defines a curve radius of between about 15 and about 21 millimeters.

14. The device recited in claim 1, wherein the pre-configured, selectively actuated curve profile defines a curve radius of between about 17 and about 19 millimeters.

15. The device recited in claim 1, wherein the hub indicates a direction of the curve profile of the elongated body.

16. The device recited in claim 15, wherein an asymmetry of the hub indicates the direction of the curve profile.

17. A device for occluding a patient's ascending aorta, comprising:
a hub;
an elongated shaft connected to the hub, the elongated shaft having opposing proximal and distal ends, the elongated body defining a plurality of lumens extending at least partially between the proximal and distal ends of the elongated shaft; and
an expandable member at least proximate the distal end of the elongated shaft, the expandable member being selectively changeable between expanded and contracted states, wherein in the expanded state, the expandable member defines a central axis offset from the elongated shaft, and has upper and lower surfaces of differing lengths.

18. The device recited in claim 17, wherein the shaft has an eccentricity between about 5% and about 36% relative to the central axis of the expandable member.

19. The device recited in claim 17, wherein the expandable member is elongated.

20. The device recited in claim 17, wherein the expandable member is substantially symmetrical about an axis separating distal and proximal portions of the expandable member, and substantially asymmetrical about the central axis.

21. The device recited in claim 17, wherein a distal tip of the elongated shaft is connected to a distal end of the expandable member, wherein an angle between the distal end of the expandable member and an upper surface of the expandable member is between about 13° and about 20°.

22. The device recited in claim 17, wherein the elongated shaft includes or is attached to a distal tip, the distal tip being substantially enclosed within the expandable member.

23. The device recited in claim 22, wherein the expandable member extends distally relative to the distal tip.

24. The device recited in claim 17, wherein at least two of said plurality of lumens are open at a distal tip of the elongated shaft.

25. The device recited in claim 17, wherein the elongated shaft has a pre-determined curve profile.

26. The device recited in claim 25, wherein the pre-determined curve profile is selectively activated.

27. The device recited in claim 17, wherein the pre-determined curve profile generally corresponds to a curve of an interior surface of the patient's aortic arch.

28. A method of delivering antegrade cardioplegic fluid to a heart of a patient, the method comprising:
introducing at least a distal end of an antegrade cardioplegic delivery catheter into a peripheral artery of the patient;
advancing the distal end of the antegrade cardioplegic delivery catheter from the peripheral artery into an ascending aorta of the heart of the patient, wherein advancing the distal end of the antegrade cardioplegic delivery catheter changing a curve profile of the delivery catheter to a predetermined curve profile configured to generally correspond to a bottom surface of an aortic arch of the patient;
occluding the ascending aorta with an occlusion device of the antegrade cardioplegic delivery catheter;
positioning a distal tip of the antegrade cardioplegic delivery catheter in a generally parallel alignment within the ascending aorta; and
delivering a fluid to the heart through a lumen of the antegrade cardioplegic delivery catheter.

29. The method recited in claim 28, further comprising after occluding the ascending aorta with the occlusion device, removing slack within the antegrade cardioplegic delivery catheter by retracting the antegrade cardioplegic delivery catheter.

30. The method recited in claim 29, wherein positioning the distal tip in a generally parallel alignment within the ascending aorta occurs following retraction of the antegrade cardioplegic delivery catheter.

31. The method recited in claim 29, wherein removing slack includes removing all slack by retracting less than about 3 centimeters of the antegrade cardioplegic delivery catheter.

32. The method recited in claim 28, wherein the predetermined curve profile has a radius of curvature between about 15 millimeters and about 21 millimeters.

33. The method recited in claim 28, wherein the occlusion device includes a balloon, wherein the antegrade cardioplegic delivery catheter is eccentric relative to the balloon.

34. The method recited in claim 28, wherein the occlusion device is asymmetrical relative to a central axis extending between distal and proximal ends of the occlusion device.

35. The method recited in claim 28, wherein the antegrade cardioplegic delivery catheter includes a core wire, the core wire being at least partially formed of a memory material, the memory material being configured to remember the predetermined curve profile upon selective activation of the memory material.

36. The method recited in claim 35, further comprising selectively activating the memory material by heating the core wire to about body temperature.
